(19) United States
(12) Patent Application Publication
(10) Pub. No.: US 2022/0273985 A1
(43) Pub. Date: Sep. 1, 2022

(54) INTERACTIVE AI COACHING-BASED MUSCULOSKELETAL EXERCISE AND REHABILITATION TRAINING SYSTEM AND METHOD

(71) Applicant: CYBERMEDIC CO., LTD., Gwangju (KR)

(72) Inventors: Ho Choon JEONG, Gunsan-si (KR); Hyun Hee LEE, Jeonju-si (KR); Sang Sea LEE, Jeonju-si (KR); Myoung Choon KIM, Iksan-si (KR)

(73) Assignee: CYBERMEDIC CO., LTD., Gwangju (KR)

(21) Appl. No.: 17/679,038

(22) Filed: Feb. 23, 2022

(30) Foreign Application Priority Data
Feb. 26, 2021 (KR) 10-2021-0026396

(51) Int. Cl. A63B 24/00; A63B 26/00; A63B 71/06; A63B 22/00; G16H 20/30; G16H 40/67

(52) U.S. Cl. CPC A63B 24/0075; A63B 24/0062; A63B 26/003; A63B 71/0622; A63B 22/0076; G16H 20/30; G16H 40/67; A63B 2230/62; A63B 2225/20; A63B 2230/06; A63B 2220/56; A63B 2022/0079

(57) ABSTRACT

An interactive AI coaching-based musculoskeletal exercise and rehabilitation training system, includes: a user state information measurement unit connected to a cloud server through a wired or wireless network and measuring state information, the state information being information about a user's exercise posture or physical condition; a display panel displaying necessary information to a user during exercise based on the state information measured by the user state information measurement unit; an exercise unit allowing a user to perform a resistance exercise or a balance exercise; a coaching engine receiving and analyzing the state information measured by the user state information measurement unit during a user's exercise using the exercise unit and providing AI-based interactive coaching information to a user through the display panel; and an expert terminal providing rehabilitation service information to a user exercising using the exercise unit through the display panel in parallel with the coaching engine.

### FIELD

The present invention relates to an interactive AI coaching-based musculoskeletal exercise and rehabilitation training system and method by which a user can be regularly prescribed at least one of resistance, aerobic, neuromotor, and flexibility exercises in a daily living space without relying on a special institution such as a clinic and can perform the prescribed exercises using one complex exercise machine installed in the same space while receiving AI coaching provided by an expert, thereby allowing exercise for improvement in physical strength of the elderly and musculoskeletal exercise and rehabilitation training for rehabilitation of patients with movement disorders to be performed in a systematic and effective manner.

### BACKGROUND

Recently, the elderly population increases with improvement in living standards and development of medical technology, and an increasing number of people have movement disorders due to industrial accidents, traffic accidents, strokes, and the like.

The elderly who want to improve their physical health or patients with movement disorders in need of musculoskeletal exercise and rehabilitation training need to be prescribed exercise or rehabilitation training that can be regularly performed at a set time every day.

Such prescribed exercise or rehabilitation training includes at least one of neuromotor, flexibility, resistance, and aerobic exercises to enhance muscle strength, endurance, sensorimotor adaptation, and cardiovascular recovery. Such exercises need to be proper for a user's condition and thus require special motor components and specialized exercise machines as well as special attention according to a specialist's prescription.

Generally, a person who wants to receive rehabilitation training has to rely on special institutions such as clinics, that is, has to go through various exercises using different exercise machines installed in a clinic under the guidance of a physical therapist.

However, it is difficult for the elderly or patients with movement disorders to travel to a clinic every day to perform prescribed exercises at a set time. In addition, even in the clinic, they have difficulty in working out through different exercises using different exercise machines.

Further, despite the fact that the elderly or patients with movement disorders have different physical conditions depending on their gender, age, and constitution, there is still no individually tailored exercise treatment based on collection and evaluation of continuous daily data, such as a user's daily condition or exercise posture, and data transformation serving an intended purpose according to kinesiological principles.

Moreover, a conventional one-way exercise coaching service using multimedia content lacks an ability to provide feedback as to whether a user performs an accurate movement or an appropriate exercise.

In addition, since such an exercise coaching service is provided on a one-off basis, a user cannot achieve steady results or changes in exercise level, can fail to maintain regular exercise routines due to lack of willpower, and can easily lose interest due to lack of motivation through competition or fellowship with others.

### SUMMARY

Embodiments of the present invention are conceived to solve such problems in the art and it is an aspect of the present invention to provide an interactive AI coaching-based musculoskeletal exercise and rehabilitation training system and method by which a user can be regularly prescribed at least one of resistance, aerobic, neuromotor, and flexibility exercises in a daily living space without relying on a special institution such as a clinic and can perform the prescribed exercises using one complex exercise machine installed in the same space while receiving AI coaching provided by an expert, thereby allowing exercise for improvement in physical strength of the elderly and musculoskeletal exercise and rehabilitation training for rehabilitation of patients with movement disorders to be performed in a systematic and effective manner.

It is another aspect of the present invention to provide an interactive AI coaching-based musculoskeletal exercise and rehabilitation training system and method by which big data related to rehabilitation programs, posture correction, posture analysis, and rehabilitation training prescriptions can be shared and used among the elderly who want to improve their physical strength or patients with movement disorders in need of musculoskeletal exercise and rehabilitation training or among doctors and rehabilitation specialists, thereby allowing users to motivate one another and to have interest in rehabilitation training. It is a further aspect of the present invention to provide an interactive AI coaching-based musculoskeletal exercise and rehabilitation training system and method which can perform isokinetic, isotonic, isometric, and fall risk evaluation while allowing a user to perform various exercises, such as exercises for each body part, whole body exercises, neuromotor exercises, flexibility exercises, resistance exercises, and aerobic exercises, at home using one complex exercise machine.

It will be understood that aspects of the present invention are not limited to the above. The above and other aspects of the present invention will become apparent to those skilled in the art from the detailed description of the following embodiments in conjunction with the accompanying drawings.

In accordance with one aspect of the present invention, an interactive AI coaching-based musculoskeletal exercise and rehabilitation training system includes: a user state information measurement unit connected to a cloud server through a wired or wireless network and measuring state information, the state information being information about a user's exercise posture or physical condition; a display panel displaying necessary information to a user during exercise based on the state information measured by the user state information measurement unit; an exercise unit allowing a user to perform a resistance exercise or a balance exercise; a coaching engine receiving and analyzing the state information measured by the user state information measurement unit during a user's exercise using the exercise unit and providing AI-based interactive coaching information to a user through the display panel; and an expert terminal providing rehabilitation service information to a user exercising using the exercise unit through the display panel in parallel with the coaching engine.

The user state information measurement unit may include: a motion detector detecting a user's posture and motion; a plantar pressure measurement unit measuring a user's plantar pressure; a heart rate measurement unit measuring a user's heart rate; and a load detector detecting a load applied to the exercise unit by a user.

The exercise unit may include at least one of: a first resistance exercise device allowing a user to perform a first resistance exercise; and a first balance exercise device allowing a user to perform a first balance exercise.

The first resistance exercise device may include a wire pulley unit generating resistance against pulling force applied to a wire by a user.

The wire pulley unit may include: a rotor rotatably mounted in a housing securely supporting the display panel, the rotor being connected to one end of the wire; and a drive unit generating resistance against pulling force applied to the wire by a user and rotating the rotor to allow the one end of the wire to be wound around the rotor when the pulling force is removed from the wire.

The first resistance exercise device may further include a pair of arm units allowing adjustment of a position and angle of the wire pulled by a user in front of the display panel.

Each of the pair of arm units may include: a rotary block disposed on one side of a housing securely supporting the display panel, the rotary block being coupled to the housing to be rotatable about a hollow vertical shaft through which the wire passes; an arm mount coupled to the rotary block to be rotatable about a hollow horizontal shaft through which the wire passes; and an arm member having one end coupled to the arm mount and provided in the form of a bar having a hollow inside to allow passage of the wire therethrough.

The exercise unit may further include: a second resistance exercise device allowing a user to perform a second resistance exercise, wherein the second resistance exercise device may include: a support placed on a floor in front of the display panel; a footrest coupled to the support and securing a user's feet thereto; a seat mounted on the support to be slidable back and forth and allowing a user to be seated thereon; and a handlebar coupled to the wire and pulled by a user.

The first balance exercise device may include a plantar pressure pad allowing a user to stand thereon and including a plantar pressure measurement unit measuring a user's plantar pressure, wherein the plantar pressure pad may further include a guidance indicator synchronized with information displayed on the display panel and indicating a position at which a user's feet are to be placed on the plantar pressure pad.

The exercise unit may further include: a second balance exercise device allowing a user to perform a second balance exercise, wherein the second balance exercise device may include a guide beam unit delivering a guide beam to guide the user to stay within a detection range of a motion detector while the motion detector detects the user's posture and motion during the second balance exercise.

The coaching engine may include: a UI/UX providing unit allowing selection between a resistance exercise mode and a balance exercise mode using the display panel; a network input/output unit allowing access to the cloud server; and a curation unit evaluating a user's exercise based on the user state information received from the user state information measurement unit and curating a training program.

The coaching engine may further include: a training information analysis unit analyzing compatibility of the training program curated by the curation unit with the user state information received from the user state information measurement unit; a training program preparation unit preparing an AI-based training prescription based on analysis by the training information analysis unit; and a training coaching unit performing training coaching according to the training prescription prepared by the training program preparation unit.

In accordance with another aspect of the present invention, a training method using the interactive AI coaching-based musculoskeletal exercise and rehabilitation training system set forth above includes: inputting user information; selecting between a resistance exercise mode and a balance exercise mode; performing a rehabilitation exercise; measuring the state information during the rehabilitation exercise; and receiving the AI-based interactive coaching information during the rehabilitation exercise.

The training method may further include: before receiving the AI-based interactive coaching information, setting whether to receive the AI-based interactive coaching information during the rehabilitation exercise.

The training method may further include: further receiving the rehabilitation service information from an expert during the rehabilitation exercise.

The interactive AI coaching-based musculoskeletal exercise and rehabilitation training system and method according to the embodiments of the present invention allow exercise for improvement in physical strength of the elderly and rehabilitation training for rehabilitation of patients with movement disorders to be performed in a systematic and effective manner using multifunctional exercise equipment for complex rehabilitation exercises such as resistance, aerobic, neuromotor, and flexibility exercises.

In addition, the interactive AI coaching-based musculoskeletal exercise and rehabilitation training system and method according to the embodiments of the present invention allow big data related to rehabilitation programs, posture correction, posture analysis, and rehabilitation training prescriptions to be shared and used among the elderly who want to improve their physical strength or patients with movement disorders in need of musculoskeletal exercise and rehabilitation training or among doctors and rehabilitation specialists, thereby generating motivation and excitement through competition or fellowship among different users and thus allowing more systematic and effective rehabilitation training.

Further, the interactive AI coaching-based musculoskeletal exercise and rehabilitation training system and method according to the embodiments of the present invention can perform isokinetic, isotonic, isometric, and fall risk evaluation while allowing a user to perform various exercises, such as exercises for each body part, whole body exercises, neuromotor exercises, flexibility exercises, resistance exercises, and aerobic exercises, at home using one complex exercise machine.

It will be understood that advantageous effects of the present invention are not limited to the above and include any advantageous effects conceivable from the features disclosed in the detailed description of the present invention or the appended claims.

### DRAWINGS

The above and other aspects, features, and advantages of the present invention will become apparent from the detailed description of the following embodiments in conjunction with the accompanying drawings:

FIG. 1 is a diagram of an interactive AI coaching-based musculoskeletal exercise and rehabilitation training system according to one embodiment of the present invention;

FIG. 2 is a block diagram of a user state information measurement unit of the interactive AI coaching-based musculoskeletal exercise and rehabilitation training system according to one embodiment of the present invention;

FIG. 3 is a schematic view of a first resistance exercise device of the interactive AI coaching-based musculoskeletal exercise and rehabilitation training system according to one embodiment of the present invention;

FIG. 4 is a view of a wire pulley unit of the first resistance exercise device according to one embodiment of the present invention;

FIG. 5 is a view of an arm unit of the first resistance exercise device according to one embodiment of the present invention;

FIG. 6 is a view illustrating a connection structure between the arm unit and the wire pulley unit of the first resistance exercise device according to one embodiment of the present invention.

FIG. 7 is a schematic view of a second resistance exercise device of the interactive AI coaching-based musculoskeletal exercise and rehabilitation training system according to one embodiment of the present invention;

FIG. 8 is a schematic view of a first balance exercise device of the interactive AI coaching-based musculoskeletal exercise and rehabilitation training system according to one embodiment of the present invention;

FIG. 9 is a block diagram of a coaching engine of the interactive AI coaching-based musculoskeletal exercise and rehabilitation training system according to one embodiment of the present invention;

FIG. 10 is a diagram illustrating a curation service provided by the coaching engine according to the embodiment.

FIG. 11 is a flowchart of a training method using the interactive AI coaching-based musculoskeletal exercise and rehabilitation training system according to one embodiment of the present invention;

FIG. 12 is a flowchart of an interactive AI coaching-based musculoskeletal exercise and rehabilitation training method using the first resistance exercise device according to one embodiment of the present invention;

FIG. 13 is a flowchart of an interactive AI coaching-based musculoskeletal exercise and rehabilitation training method using the second resistance exercise device according to one embodiment of the present invention;

FIG. 14 is a flowchart of an interactive AI coaching-based musculoskeletal exercise and rehabilitation training method using the first balance exercise device according to one embodiment of the present invention; and

FIG. 15 is a flowchart of an interactive AI coaching-based musculoskeletal exercise and rehabilitation training method using the second balance exercise device according to one embodiment of the present invention.

### DETAILED DESCRIPTION

Hereinafter, exemplary embodiments of the present invention will be described in detail with reference to the accompanying drawings. Like components will be denoted by like reference numerals throughout the specification. Description of known functions and constructions which may unnecessarily obscure the subject matter of the present invention will be omitted. For the same reason, some components are exaggerated, omitted, or schematically illustrated in the accompanying drawings.

Further, throughout the specification, the terms “comprises,” “comprising,” “includes,” and/or “including,” when used in this specification, specify the presence of stated elements, but do not preclude the presence or addition of one or more other elements. Moreover, throughout the specification, when an element is referred to as being placed “on” another element, the element may be placed on an upper surface or a lower surface of the other element, and is not necessarily placed above the other element with respect to the direction of gravity.

First, an interactive AI coaching-based musculoskeletal exercise and rehabilitation training system according to one embodiment of the present invention will be described with reference to FIG. 1.

FIG. 1 is a diagram of an interactive AI coaching-based musculoskeletal exercise and rehabilitation training system according to one embodiment of the present invention.

The interactive AI coaching-based musculoskeletal exercise and rehabilitation training system according to this embodiment may include a user state information measurement unit 200, a display panel 300, an exercise unit, a coaching engine 600, and an expert terminal 700.

The user state information measurement unit 200 may be connected to a cloud server 100 that provides recording and storage of user DB information, data management, and content services through a wired or wireless network. The user state information measurement unit 200 may be installed in a house, and may measure state information for prescription of exercises for improvement in physical strength of the elderly and rehabilitation training for patients with movement disorders. Here, the state information may include information about a user's exercise posture or a user's physical condition.

The display panel 300 may provide various types of necessary information to a user in real time during exercise using an interactive chatbot based on the user state information measured by the user state information measurement unit 200.

The exercise unit may be electrically or mechanically detachably coupled to the display panel 300, and may allow a user to perform exercises prescribed for them. The exercise unit may allow a user to perform a complex exercise including at least one resistance exercise or at least one balance exercise. Using the exercise unit, a user may perform at least one selected from among resistance, aerobic, neuromotor, and flexibility exercises.

During a user's exercise and rehabilitation training using the exercise unit, the coaching engine 600 may receive and analyze the user state information measured by the user state information measurement unit 200 and may provide AI-based interactive coaching information to the user through the display panel 300.

The expert terminal 700 may provide a user exercising using the exercise unit with rehabilitation service information corresponding to content and services related to a rehabilitation program, posture correction, posture and movement analysis, and a rehabilitation training prescription, in parallel with the coaching engine 600. The expert terminal 700 may be operated by a doctor or a rehabilitation specialist who selectively charges a fee for their services.

In this system, using a subscription service, a user can be regularly provided with an exercise prescription consisting of at least one of resistance, aerobic, neuromotor, and flexibility exercises, which need to be performed at a set time every day using the interactive smart complex exercise unit. In addition, during rehabilitation training at home, a user can receive AI-based feedback from the AI-based interactive coaching engine 600 and can be easily and selectively provided with content and services related to a rehabilitation program, posture correction, posture and movement analysis, and a rehabilitation training prescription from the expert terminal 700. Accordingly, a user can perform systematic and effective rehabilitation training at a set time every day.

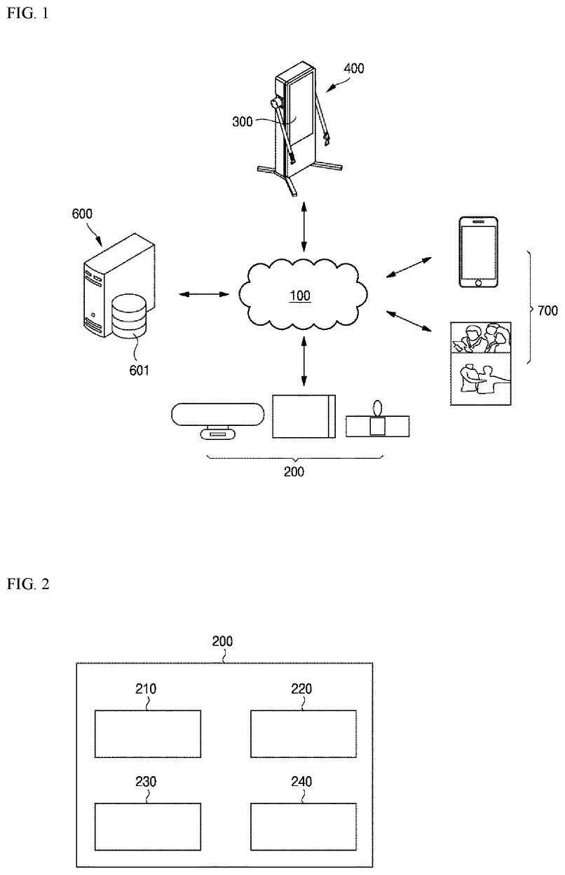

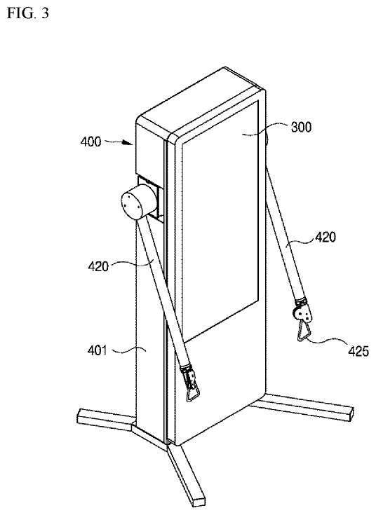

FIG. 2 is a block diagram of the user state information measurement unit of the interactive AI coaching-based musculoskeletal exercise and rehabilitation training system according to one embodiment of the present invention.

Referring to FIG. 2, the user state information measurement unit 200 may include a motion detector 210, a plantar pressure measurement unit 220, a heart rate measurement unit 230, and a load detector 240.

The motion detector 210 serves to detect a user's posture and motion during exercise, and may be disposed above the display panel 300. The motion detector 210 may measure movement of whole body joints of a user exercising in front of the display panel 300. The motion detector 210 may be a 3D motion camera.

The plantar pressure measurement unit 220 serves to measure the plantar pressure of a user during exercise, and may be disposed on a floor in front of the display panel 300. The plantar pressure measurement unit 220 may measure the distribution of plantar pressure and the trajectory of the center of pressure (COP) of a user during exercise through measurement of the plantar pressure of the user, and may analyze postural balance of the user during exercise based on the measurement results. The plantar pressure measurement unit 220 may be an array of force sensing resistors (FSRs) arranged in a grid pattern on a plantar pressure pad 510 of a first balance exercise device 500 described below.

The heart rate measurement unit 230 serves to measure the heart rate of a user during exercise. The heart rate measurement unit 230 may be a heart rate sensor known in the art. Such a heart rate sensor may be mounted on a handle 425 of a first resistance exercise device 400 described below, or may be worn on the wrist of a user before exercise, like a bracelet.

The load detector 240 serves to detect a load applied to the exercise unit by a user during exercise. The load detector 240 may be provided to the exercise unit, specifically to a drive unit 412 of the first resistance exercise device 400 described below. The load detector 240 may be a high-resolution encoder.

The user state information measurement unit 200 may further include a speed detector. The speed detector serves to detect a user's speed during exercise, and may be provided to the drive unit 412 of the exercise unit described below. The speed sensor may be an encoder.

The display panel 300 may display information necessary for a user during exercise based on the user state information measured by the user state information measurement unit 200.

That is, the display panel 300 may inform a user performing exercise of the user state information measured by the user state information measurement unit 200, the AI-based interactive coaching information provided by the coaching engine 600, and the rehabilitation service information provided by the expert terminal 700, along with various related content.

The display panel 300 may be a tower type, which is erected on a floor, or a wall-mounted type.

Next, the exercise unit according to one embodiment of the present invention will be described in detail with reference to FIG. 3 to FIG. 6.

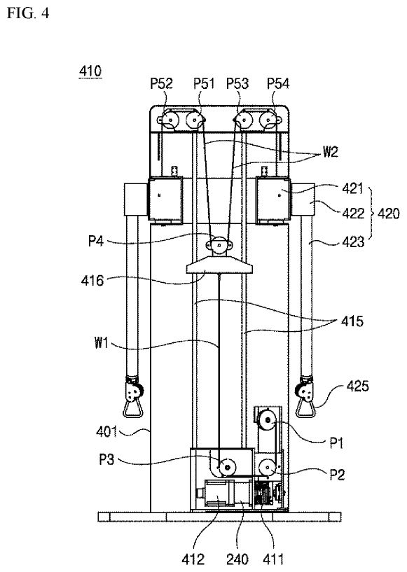

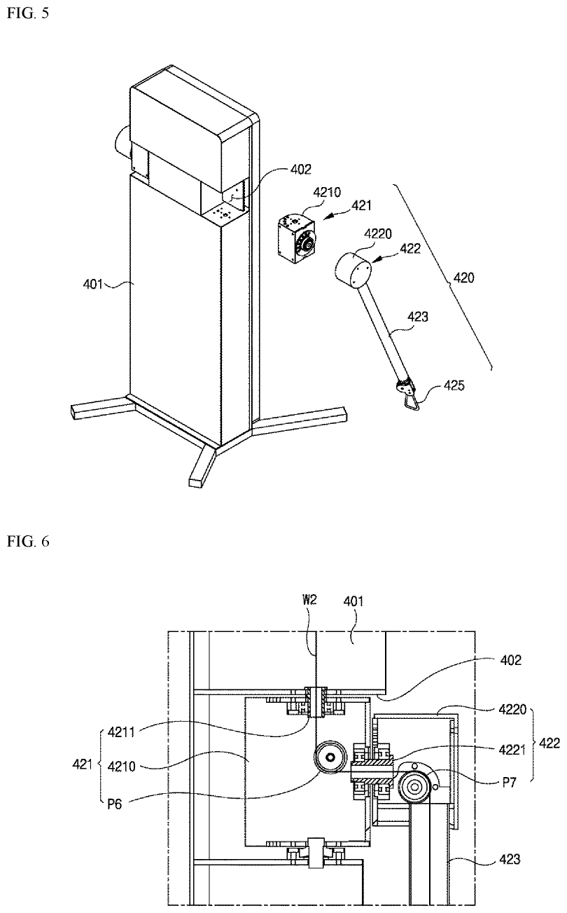

FIG. 3 is a schematic view of a first resistance exercise device of the interactive AI coaching-based musculoskeletal exercise and rehabilitation training system according to one embodiment of the present invention, FIG. 4 is a view of a wire pulley unit of the first resistance exercise device according to one embodiment of the present invention, FIG. 5 is a view of an arm unit of the first resistance exercise device according to one embodiment of the present invention, and FIG. 6 is a view illustrating a connection structure between the arm unit and the wire pulley unit of the first resistance exercise device according to one embodiment of the present invention.

The exercise unit may include a first resistance exercise device 400.

The first resistance exercise device 400 allows a user to perform a first resistance exercise based on the AI-based interactive coaching information or the rehabilitation service information displayed on the display panel 300 through the coaching engine 600 or the expert terminal 700.

The first resistance exercise device 400 may include a wire pulley unit 410 and an arm unit 420.

The wire pulley unit 410 may generate resistance against pulling force applied to a wire by a user.

The wire pulley unit 410 may include: a rotor 411 rotatably mounted in a housing 401 securely supporting the display panel 300, the rotor being connected to one end of the wire; and a drive unit 412 generating resistance against pulling force applied to the wire by a user and rotating the rotor 411 to allow the one end of the wire to be wound around the rotor 441 when the pulling force is removed from the wire. The drive unit 412 may be a BLDC motor known in the art.

Specifically, in the wire pulley unit 410, the rotor 411 to which the one end of the wire is connected is disposed at an inner lower portion of the housing 401 and a handle 425 to which the other end of the wire is connected is coupled to an end of the arm unit 420. The rotor 411 may have a wire winding groove formed around an outer surface thereof. When a user's pulling force is removed from the handle 425, the rotor 411 is rotated by driving force of the driving unit 412 to allow the wire to be wound around the wire winding groove. When a user pulls the handle 425 with a force stronger than the driving force of the driving unit 412, the rotor 411 is rotated in the reverse direction to allow the wire to be unwound from the wire winding groove.

The drive unit 412 may be provided with the load detector 240 as the user state information measurement unit 200. The load detector 240 may detect a load applied to the driving unit 412. That is, the load detector 240 may detect a user's pulling force stronger than a predetermined driving force of the drive unit 412.

In addition, the wire of the wire pulley unit 410 may include a first wire W1 and a second wire W2, wherein the wire pulley unit 410 may further include a wire connector 416 connecting the first wire W1 to the second wire W2.

The first wire W1 may have one end connected to the rotor 411 and the other end connected to the wire connector 416 after being redirected in an upward direction of the housing 401 by passing over a first pulley P1, a second pulley P2, and a third pulley P3 in the stated order.

Referring to FIG. 4, the drive unit 412 may be disposed at a center of a bottom of the housing 401. Since the drive unit 412, which is relatively heavy, is disposed at the center of the bottom of the housing 401, the center of gravity of the housing 401 securely supporting the display panel 300 can be stabilized during the first resistance exercise, thereby allowing increase in positional stability of the housing 401.

Referring to FIG. 4, as the drive unit 412 is disposed at the center of the bottom of the housing 401, the rotor 411 is disposed to be offset to one side (to the right in the drawing) from a vertical centerline of the housing 401. Accordingly, there is a need to guide the first wire W1 connected to the rotor 411 to be in line with the vertical centerline of the housing 401. That is, positional stability of the housing 401 can be ensured by disposing the wire, which is tensioned by a load applied by a user or by the driving force of the drive unit 412, on the vertical center line of the housing 401. To this end, in this embodiment, a lower section of the first wire W1 connected to the rotor 411 is guided to the vertical center line of the housing 401 using the first pulley P1, the second pulley P2, and the third pulley P3 such that the first wire W1 can be substantially in line with the vertical center line of the housing 401.

Specifically, the first pulley P1 is disposed above the rotor 411 in a tilted manner such that the first wire W1 extending upwards from the wire winding groove of the rotor 411 is redirected by the first pulley P1 to extend downwards toward a rotational center of the rotor. The second pulley P2 is disposed below the first pulley P1 such that the first wire W1 redirected toward the rotational center of the rotor 411 by the first pulley P1 is redirected by the second pulley P2 to horizontally extend toward the vertical center line of the housing 401. The third pulley P3 is disposed above the drive unit 412 such that the first wire W1 redirected toward the vertical centerline of the housing 401 by the second pulley P2 is redirected by the third pulley P3 to extend upwards toward the wire connector 416. As such, since the drive unit 412, which is relatively heavy, is disposed at the center of the bottom of the housing 401 and the first wire W1 connected to the rotor 411 offset from the vertical centerline of the housing 401 is disposed in the vertical centerline of the housing 401, it is possible to guarantee positional stability of the housing 401, that is, to prevent the housing 401 from turning or falling, even when strong tension is applied to the wire by a load applied by a user or by the driving force of the drive unit 412.

The second wire W2 has a middle section coupled to a fourth pulley P4 disposed on the wire connector 416, a left section connected to a left arm unit, and a right section connected to a right arm unit.

Specifically, the left section of the second wire W2 extending from the fourth pulley P4 toward an upper portion of the housing 401 is connected to the left arm unit after being redirected toward a lower left portion of the housing 401 by sequentially passing over fifth left pulleys P51, P52. The right section of the second wire W2 extending from the fourth pulley P4 toward an upper portion of the housing 401 is connected to the right arm unit after being redirected toward a lower light portion of the housing 401 by sequentially passing over fifth right pulleys P53, P54. Since the second wire W2 is coupled at the middle section to the fourth pulley P4 on the wire connector 416, the left arm unit connected to the left section of the second wire W2 may be synchronized in operation with the right arm unit connected to the right section of the second wire W2.

The wire pulley unit 410 may further include a connector guide 415. The connector guide 415 may extend through the wire connector 416. The connector guide 415 may include a pair of connector guides 415 disposed on opposite sides with respect to the vertical centerline of the housing 401. The pair of connector guides 415 may extend in a vertical direction of the housing 401. Accordingly, the connector guide 415 can guide upward and downward movement of the wire connector 416.

The arm unit 420 allows adjustment of the position and angle of the wire pulled by a user exercising in front of the display panel 300.

The arm unit 420 may include a pair of arm units disposed on opposite sides of the housing 401, specifically a left arm unit connected to the left section of the second wire W2 and a right arm unit connected to the right section of the second wire W2.

Each of the pair of arm units 420 may include a rotary block 421, an arm mount 422, and an arm member 423.

The rotary block 421 is disposed in a mounting space 402 defined on a side surface of the housing 401, and may be coupled to the housing 401 to be rotatable about a hollow vertical shaft 4211 through which the second wire W2 passes.

Specifically, the rotary block 421 may include a rotary block body 4210 disposed in the mounting space 402 of the housing 401 and having an empty inside, the vertical shaft 4211 rotatably connecting the rotary block body 4210 to the mounting space 402, and a sixth pulley P6 disposed in an inner space of the rotary block body 4210. Accordingly, the end of the second wire W2 extending downwards along a side of the housing 401 after passing over the fifth left pulleys P53, P54 of FIG. 4 extends into the rotary block body 4210 through a hollow portion of the vertical shaft 4211 and then passes over the sixth pulley P6 to be redirected towards the arm mount 422 disposed outside the rotary block body 210. In this way, when the rotary block body 4210 is rotated about the vertical shaft 4211, the second wire W2 can be prevented from twisting upon passing through the inner space of the rotary block body 4210, thereby allowing tension to be stably transmitted to the second wire W2 during the first resistance exercise.

The arm mount 422 may be coupled to the rotary block 421 to be rotatable about a hollow horizontal shaft 4221 through which the second wire W2 passes.

Specifically, the arm mount 422 may include an arm mount body 4220 coupled to an outer surface of the rotary block body 4210 and having an empty inside, a horizontal shaft 4221 connecting the rotary block body 4210 to the arm mount body 4220, and a seventh pulley P7 disposed in an inner space of the arm mount body 4220. Accordingly, the end of the second wire W2 extending in the horizontal direction after passing over the sixth pulley P6 extends into the arm mount body 4220 through a hollow portion of the horizontal shaft 4221 and then passes over the seventh pulley P7 to be redirected in a longitudinal direction of the arm member 423. In this way, when the arm mount body 4220 is rotated about the horizontal shaft 4211, the second wire W2 can be prevented from twisting upon passing through the inner space of the arm mount body 4220, thereby allowing tension to be stably transmitted to the second wire W2 during the first resistance exercise.

The arm member 423 is coupled at one end thereof to the arm mount 422, and may be provided in the form of a bar having a hollow inside. In addition, the arm member 423 is provided at the other end thereof with an eighth pulley on which the end of the second wire W2 is supported.

Here, the handle 425 with the end of the second wire W2 coupled thereto may be caught and supported by the arm member 423 due to resistance generated by the wire pulley unit 410. That is, when a user's pulling force is removed from the handle 425, the handle 425 can be caught and supported by the eighth pulley at the other end of the arm member 423 due to tension applied to the second wire W2 by the driving force of the drive unit 412.

As such, the arm member 423 connected to the housing 401 via the rotary block 421 and the arm mount 422 can be rotated with respect to the housing 401 like a human arm, thereby allowing a user to freely adjust the position and angle of the handle 425 upon pulling the wire using the handle 425. That is, a user can selectively perform various types of first resistance exercise, such as high pulley exercise and low pulley exercise, depending on the position of the handle 425. Furthermore, the arm member 423 allows a user to maintain the proper position and angle of the wire required for a second resistance exercise using a second resistance exercise device 450 described below.

The first resistance exercise device 400 may further include a controller. The controller controls the wire pulley unit 410 based on the coaching information provided by the coaching engine 600 or the rehabilitation service information provided by the expert terminal 700. That is, it is possible to adjust and set the driving force of the drive unit 412 of the first resistance exercise device 400 through the coaching engine 600 and the expert terminal 700.

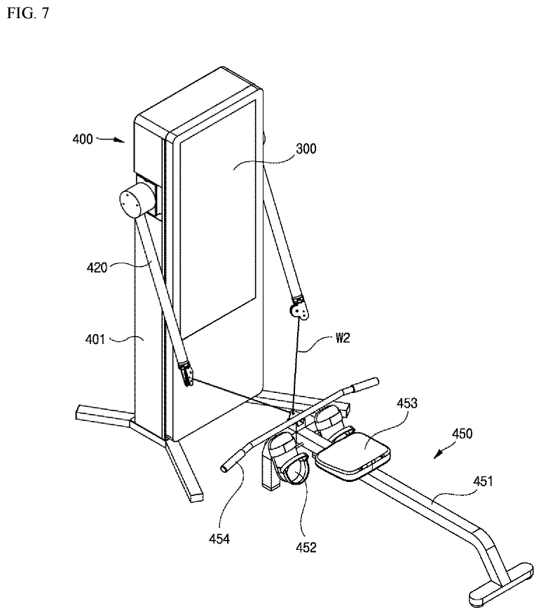

FIG. 7 is a schematic view of a second resistance exercise device of the interactive AI coaching-based musculoskeletal exercise and rehabilitation training system according to one embodiment of the present invention.

The exercise unit may further include a second resistance exercise device 450.

The second resistance exercise device 450 allows a user to perform a second resistance exercise based on the AI-based interactive coaching information or the rehabilitation service information displayed on the display panel 300 through the coaching engine 600 or the expert terminal 700.

The second resistance exercise device 450 may be placed against a side surface of the first resistance exercise device 400 when not in use, and may be disposed at a desired location on a floor in front of the first resistance exercise device 400 when in use.

The second resistance exercise device 450 may include a support 451 disposed on the floor in front of the display panel 300, a footrest 452 coupled to the support 451 and securing a user's feet thereto, a seat 453 mounted on the support 451 to be slidable back and forth and allowing a user to sit thereon, and a handlebar 454 coupled to the wire and pulled by a user. Using the second resistance exercise device 450, a user may perform a rowing exercise corresponding to the second resistance exercise.

The handle 425 of the first resistance exercise device 400 or both ends of the second wire W2 with the handle 425 removed therefrom may be connected to the handlebar 454. Accordingly, during the second resistance exercise using the second resistance exercise device 450, the wire pulley unit 410 of the first resistance exercise device 400 can provide resistance needed for a user to perform the second resistance exercise. That is, both resistance needed for the first resistance exercise and resistance needed for the second resistance exercise can be generated using one wire pulley unit 410.

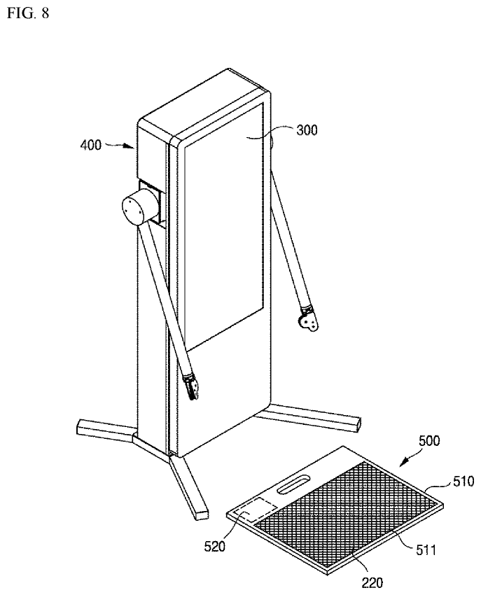

FIG. 8 is a schematic view of a first balance exercise device of the interactive AI coaching-based musculoskeletal exercise and rehabilitation training system according to one embodiment of the present invention.

The exercise unit may further include a first balance exercise device 500.

The first balance exercise device 500 allows a user to perform a first balance exercise based on the AI-based interactive coaching information or the rehabilitation service information displayed on the display panel 300 through the coaching engine 600 or the expert terminal 700.

The first balance exercise device 500 may include a plantar pressure pad 510.

The plantar pressure pad 510 allows a user to stand thereon, and may include the plantar pressure measurement unit 220 as the user state information measurement unit 200. The plantar pressure measurement unit 220 serves to measure the distribution of plantar pressure and the trajectory of the center of pressure (COP) through measurement of the plantar pressure of a user performing the first balance exercise and to analyze postural balance of the user based on the measurement results. The plantar pressure measurement unit 220 may be an array of FSRs arranged in a grid pattern on the plantar pressure pad 510.

In addition, the plantar pressure pad 510 may further include a guidance indicator 511. The guidance indicator 511 serves to indicate a position on the plantar pressure pad 510 at which a user's feet are to be placed through synchronization with the AI-based interactive coaching information or the rehabilitation service information displayed on the display panel 300. Accordingly, a user can intuitively know where to actually place their feet using the guidance display unit 511 of the plantar pressure pad 510 as well as the information displayed on the display panel 300, and thus can perform the first balance exercise with improved accuracy and speed. The guidance display unit 511 may be an array of LED lamps corresponding to the multiple FSRs arranged in a grid pattern on the plantar pressure pad 510.

The foot plantar pressure pad 510 may further include a control module 520.

The control module 520 may process a plantar pressure signal measured by the plantar pressure measurement unit 220 and a signal to turn the guidance indicator 511 on/off and may control communication with external devices.

That is, the control module 520 may process the plantar pressure signal measured by the plantar pressure measurement unit 220 and the signal to turn the guidance display unit 511 on/off and then may send the processed signals to the cloud server 100 or to the display panel 300, the coaching engine 600, and the expert terminal 700. In addition, the control module 520 may receive a signal to turn the guidance display unit 511 on/off, which is set according to the coaching information or the rehabilitation service information provided by the coaching engine 600 or the expert terminal 700.

The first balance exercise using the first balance exercise device 500 allows evaluation of the risk of fall, visual sense, vestibular sense, and somatic sense of a user through measurement of the user's sense of balance, and, furthermore, allows evaluation of limits of stability or dynamic balance. In addition, the first balance exercise can improve neuromotor function of a user.

The exercise unit may further include a second balance exercise device.

The second balance exercise device allows a user to perform a second balance exercise based on the AI-based interactive coaching information or the rehabilitation service information displayed on the display panel 300 through the coaching engine 600 or the expert terminal 700.

For example, the second balance exercise may be a flexibility exercise such as stretching, and allows a user to repeatedly perform various types of second balance exercise based on VR exercise postures, exercise scenarios, and content information needed for a user to take a specific posture, which are displayed on the display panel 300 through the coaching engine 600 or the expert terminal 700. Here, the motion detector 210 may detect, in real time, a user's posture and motion during the second balance exercise, thereby allowing analysis of differences between the detected user's posture and motion and a standard posture and motion displayed on the display panel 300 through the coaching engine 600 or the expert terminal 700.

In addition, the second balance exercise device may include a guide beam unit.

When the motion detector 210 detects a user's posture and motion during the second balance exercise, the guide beam unit delivers a guide beam to guide the user to stay within the detection range of the motion detector 210. That is, the guide beam unit prevents a user performing exercise from moving out of the detection range of the motion detector 210 by illuminating a region corresponding to the detection range of the motion detector 210 with the guide beam. Since the guide beam unit illuminates a region around a user performing exercise with the guide beam visible to the human eye, the user can take a standard posture or a standard motion within the region illuminated with the guide beam, thereby improving the effects of the second balance exercise.

As such, the complex exercise unit including the first resistance exercise device 400, the second resistance exercise device 450, the first balance exercise device 500, and the second balance exercise device allows a user to perform the first resistance exercise, the second resistance exercise, the first balance exercise, and the second balance exercise separately or in combination based on the AI-based interactive coaching information or the rehabilitation service information displayed on the display panel 300 through the coaching engine 600 or the expert terminal 700.

In addition, since the state information measured by the user state information measurement unit 200, which corresponds to a user's posture and motion, and the coaching information or the rehabilitation service information provided by the coaching engine 600 or the expert terminal 700, which corresponds to a standard posture and a standard motion, are displayed as an overlay on the display panel 300 during the first resistance exercise, the second resistance exercise, the first balance exercise, and the second balance exercise using the exercise unit, the user can receive visual feedback related to accuracy of their posture and motion, which can be considered as effective as personal training (PT). For example, the display panel 300 may operate in a mirror mode and a display mode. In the mirror mode, the display panel 300 may function as a mirror to reflect an image of a user's current posture and motion during exercise. In the display mode, the display panel 300 may display various types of information as described above.

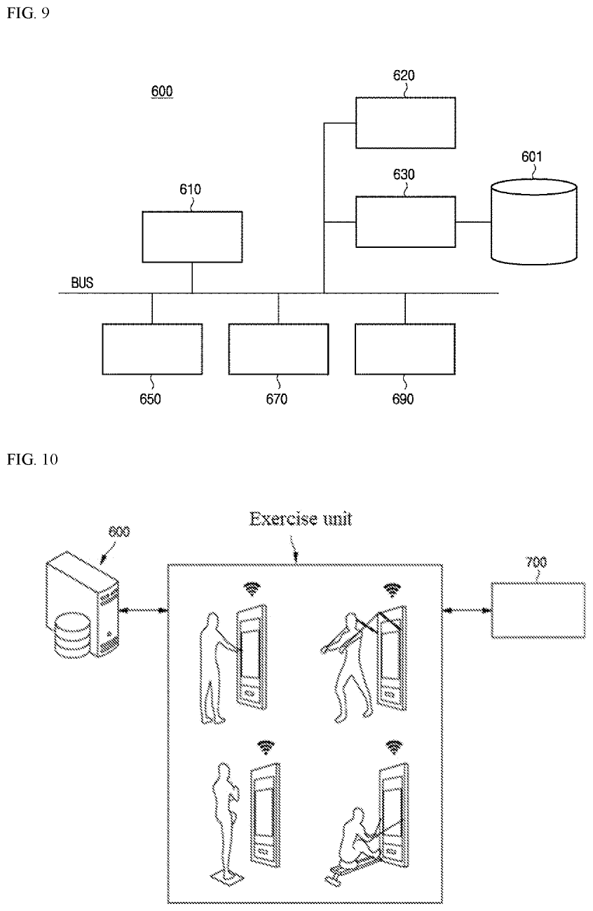

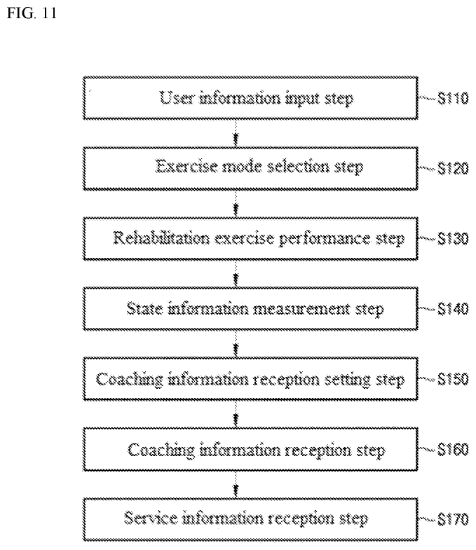

Next, the coaching engine of the interactive AI coaching-based musculoskeletal exercise and rehabilitation training system according to one embodiment of the present invention will be described with reference to FIG. 9 and FIG. 10.

FIG. 9 is a block diagram of the coaching engine of the interactive AI coaching-based musculoskeletal exercise and rehabilitation training system according to one embodiment of the present invention and FIG. 10 is a diagram illustrating a curation service provided by the coaching engine according to the embodiment.

Referring to FIG. 9, the coaching engine 600 according to this embodiment may include a UI/UX providing unit 610, a network input/output unit 620, and a curation unit 630.

The UI/UX providing unit 610 allows selection between exercise modes.

Specifically, the UI/UX providing unit 610 allows a user to intuitively select a specific exercise mode such as a resistance exercise mode and a balance exercise mode by touching the display panel 300.

The network input/output unit 620 allows access to the cloud server 100. Specifically, the network input/output unit 620 allows a user, an assistant, or an expert can intuitively access the cloud server 100 by touching the display panel 300.

The curation unit 630 serves to evaluate a user's exercise and to curate a training program based on the user state information received from the user state information measurement unit 200.

The coaching engine 600 may further include a training information analysis unit 650, a training program preparation unit 670, and a training coaching unit 690.

The training information analysis unit 650 serves to analyze compatibility of the training program curated by the curation unit 630 with the user state information received from the user state information measurement unit 200.

The training program preparation unit 670 serves to prepare an AI-based training prescription based on analysis by the training information analysis unit 650.

The training coaching unit 690 serves to perform training coaching according to the training prescription prepared by the training program preparation unit 670. That is, the training coaching unit 690 may provide a user with AI-based training coaching information including real-time VR content through the display panel 300 according to the training prescription prepared by the training program preparation unit 670.

As such, the coaching engine 600 helps a user have fun during exercise through provision of the AI-based training coaching information including VR content through the display panel 300, thereby ensuring more effective rehabilitation training. For example, the AI-based training coaching information may be provided to a user using a real-time interactive chatbot.

The coaching engine 600 may further include a database 601. The database 601 serves to store and record the AI-based training coaching information and the rehabilitation service information provided by the expert terminal 700, as well as the user state information measured by the user state information measurement unit 200.

Referring to FIG. 10, the coaching engine 600 may curate various resistance exercises or balance exercises using the exercise unit and the expert terminal 700 allows simultaneous participation of a third party, such as an assistant, and a doctor/rehabilitation specialist.

For example, the first resistance exercise using the first resistance exercise device 400 is a strength exercise for a specific body part, and may include resistance exercises targeting a user's trunk, shoulder, elbow, hip joint, and knee, which are performed by pulling the wire at different angles or speeds. During the first resistance exercise, isokinetic, isotonic, and isometric evaluation for each body part may be performed and AI-based training coaching may be conducted for each body part through muscle strength, muscle hypertrophy, muscle endurance, pain, muscle coordination, and muscle mobility exercises and motion analysis.

The second resistance exercise using the second resistance exercise device 450 is a whole body strength exercise, and may include whole body exercises performed by pulling the wire at different angles or speeds. During the second resistance exercise, isokinetic, isotonic, and isometric evaluation for a user's whole body may be performed and training coaching may be conducted through whole body exercise training and motion analysis.

The first balance exercise using the first balance exercise device 500 is an exercise performed to improve a user's sense of balance. During the first balance exercise, the risk of fall may be assessed through evaluation of a user's sense of balance and AI-based training coaching may be conducted through balance exercise training, a balance exercise game, and motion analysis.

The second balance exercise using the second balance exercise device is a posture and motion recognition exercise. Upon detection of a user's posture and motion by the motion detector 210, the user's posture and motion in front and side view may be evaluated through posture and motion analysis and AI-based training coaching may be conducted through posture exercise training, a posture exercise game, and motion analysis.

Next, a training method using the interactive AI coaching-based musculoskeletal exercise and rehabilitation training system according to one embodiment of the present invention will be described.

FIG. 11 is a flowchart of the training method using the interactive AI coaching-based musculoskeletal exercise and rehabilitation training system according to one embodiment of the present invention.

The training method using the interactive AI coaching-based musculoskeletal exercise and rehabilitation training system according to this embodiment may include a user information input step S110, an exercise mode selection step S120, a rehabilitation exercise performance step S130, a state information measurement step S140, and a coaching information reception step S160.

In the user information entry step S110, user information is input.

That is, a user may contact and access the cloud server 100 by inputting predetermined access information on the display panel 300. In addition, a third party, such as an assistant, or an expert may contact and access the cloud server 100 by inputting the predetermined information on the expert terminal 700.

In the exercise mode selection step S120, a specific exercise mode is selected.

That is, a user, an assistant, or an expert may select between various resistance exercise or balance exercise modes corresponding to the input user information.

In the rehabilitation exercise performance step S130, a rehabilitation exercise is performed.

That is, upon selecting an exercise mode, a user may perform various rehabilitation exercises using the exercise unit based on basic information displayed on the display panel 300.

In the state information measurement step S140, user state information during the rehabilitation exercise is measured.

That is, the information about the state of a user performing the rehabilitation exercise using the exercise unit is measured by the user state information measurement unit 200.

In the coaching information reception step S160, the AI-based interactive coaching information is provided during the rehabilitation exercise.

That is, during the rehabilitation exercise using the exercise unit, the user state information measured by the user state information measurement unit 200 is analyzed and, based on this analysis, the AI-based interactive coaching information including various VR content is provided to a user through the display panel 300.

Accordingly, a user can effectively perform various rehabilitation exercises using the exercise unit with the help of the user state information displayed in real time on the display panel 300 and the real-time AI-based interactive coaching information by the coaching engine 600, which includes various VR content. In addition, the coaching engine 600 may apply feedback to the real-time coaching information based on the user state information changing in real time during the rehabilitation exercise.

The training method using the interactive AI coaching-based musculoskeletal exercise and rehabilitation training system according to this embodiment may further include a coaching information reception setting step S150.

In the coaching information reception setting step S150, whether to receive the AI-based interactive coaching information during the rehabilitation exercise is set.

That is, a user may set whether to receive the AI-based interactive coaching information from the coaching engine 600 by touching the display panel 300. That is, basically, a user may perform the rehabilitation exercise in a self-directed manner based on the basic information displayed on the display panel 300, or, if necessary, the user may be optionally provided with the AI-based interactive coaching information from the coaching engine 600.

The training method using the interactive AI coaching-based musculoskeletal exercise and rehabilitation training system according to this embodiment may further include a service information reception step S170.

The service information reception step S170 may be performed after the coaching information reception step S160 or in parallel with the coaching information reception step S160. In the service information reception step S170, the rehabilitation service information is further provided by an expert during the rehabilitation exercise.

That is, a user may set whether to further receive the rehabilitation service information from the expert by touching the display panel 300. In response to a user sending a signal requesting the rehabilitation service information to the expert terminal 700, the expert or an assistant may provide the rehabilitation service information to the user after accessing the cloud server 100 using the expert terminal 700. As a result, the user can be provided with the rehabilitation service information in parallel with the AI-based interactive coaching information from the coaching engine 600.

Next, rehabilitation exercises in different exercise modes according to one embodiment of the present invention will be described.

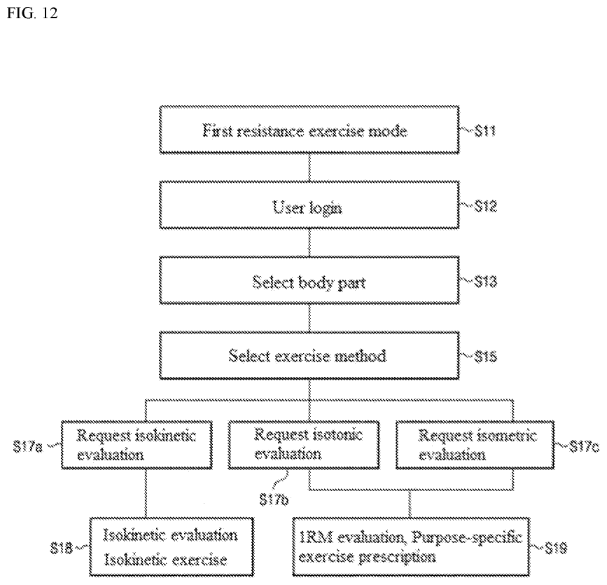

FIG. 12 is a flowchart of an interactive AI coaching-based musculoskeletal exercise and rehabilitation training method using the first resistance exercise device according to one embodiment of the present invention.

Referring to FIG. 12, when a user touches and selects a UI/UX corresponding to a first resistance exercise mode on the display panel 300 (S11), a user login process may be performed (S12). When the user touches and selects one of body parts for the first resistance exercise device-specific body parts provided by the UI/UX providing unit 610 (S13), the user may select between various exercise methods corresponding to the selected body part (S15).

When the user requests isokinetic evaluation for each body part (S17a), the user may perform an isokinetic exercise while receiving isokinetic evaluation and the AI-based interactive coaching information (S18).

When the user requests isotonic or isometric evaluation for each body part (S17b or 517c), estimation of 1 RM (the maximum amount of weight that a person can possibly lift for one repetition) may be performed and an exercise prescription suitable for an intended purpose may be provided S19.

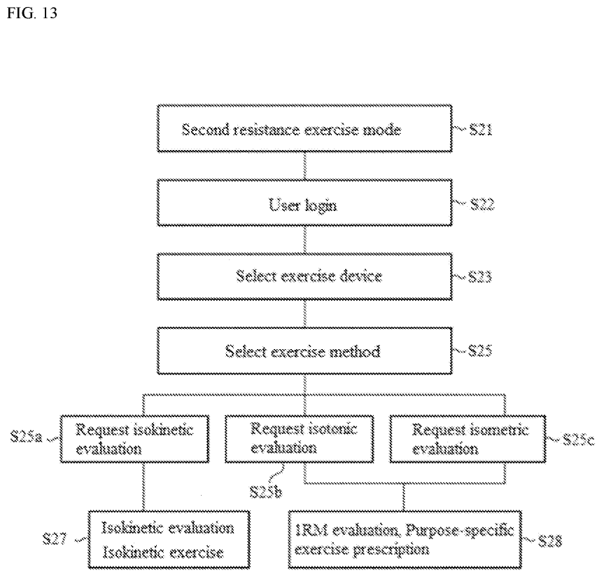

FIG. 13 is a flowchart of an interactive AI coaching-based musculoskeletal exercise and rehabilitation training method using the second resistance exercise device according to one embodiment of the present invention.

Referring to FIG. 13, when a user touches and selects a UI/UX corresponding to a second resistance exercise mode on the display panel 300 (S21), a user login process may be performed (S22). When the user touches and selects the second resistance exercise device provided by the UI/UX providing unit 610 (S23), the user may select between various exercise methods corresponding to the exercise device (S24).

When the user requests isokinetic evaluation for each body part (S25a), the user may perform an isokinetic exercise while receiving isokinetic evaluation and the AI-based interactive coaching (S27).

When the user requests isotonic or isometric evaluation for each body part (S25b or S25c), estimation of 1 RM may be performed and an exercise prescription suitable for an intended purpose may be provided (S28).

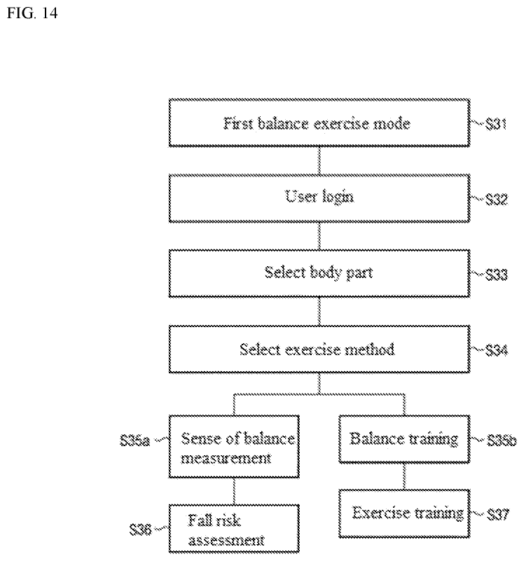

FIG. 14 is a flowchart of an interactive AI coaching-based musculoskeletal exercise and rehabilitation training method using the first balance exercise device according to one embodiment of the present invention.

Referring to FIG. 14, when a user touches and selects a UI/UX corresponding to a first balance exercise mode on the display panel 300 (S31), a user login process may be performed (S32). When the user touches and selects the first balance exercise device provided by the UI/UX providing unit 610 (S33), the user may select between various exercise methods corresponding to the exercise device (S34).

When the user selects sense of balance measurement (S35a), assessment of the risk of fall may be performed (S36).

When the user selects balance training (S35b), AI-based interactive training may be performed (S37).

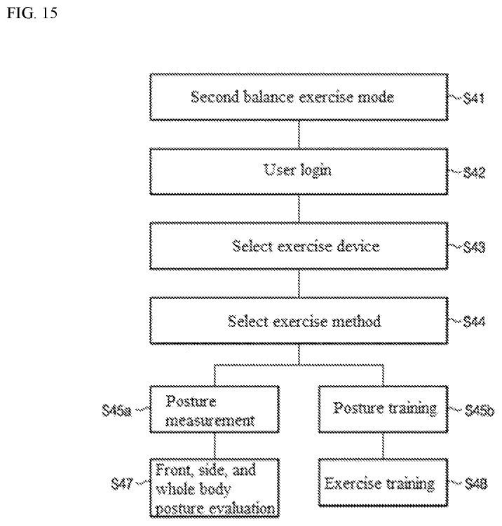

FIG. 15 is a flowchart of an interactive AI coaching-based musculoskeletal exercise and rehabilitation training method using the second balance exercise device according to one embodiment of the present invention.

Referring to FIG. 15, when a user touches and selects a UI/UX corresponding to a second balance exercise mode on the display panel 300 (S41), a user login process may be performed (S42). When the user touches and selects the second balance exercise device provided by the UI/UX providing unit (S43), the user may select between various exercise methods corresponding to the exercise device (S44).

When the user selects posture measurement (S45a), analysis and evaluation of the user's front, side, and whole body postures during exercise may be performed (S47).

When the user selects posture training (S45b), AI-based interactive training may be performed (S48).

As such, the AI-based interactive coaching engine 600 allows a user to perform various resistance exercises or balance exercises separately or in combination using preset information in the complex exercise unit with just one user log in process. In addition, the AI-based interactive coaching engine 600 can provide an effective exercise prescription for the elderly or users with movement disorders.

Although some embodiments have been described herein, it should be understood that these embodiments are provided for illustration only and are not to be construed in any way as limiting the present invention. Further, it should be understood by those skilled in the art that various modifications, changes, and alterations can be made by those skilled in the art without departing from the spirit and scope of the present invention.

1. An interactive AI coaching-based musculoskeletal exercise and rehabilitation training system comprising:
a user state information measurement unit connected to a cloud server through a wired or wireless network and measuring state information, the state information being information about a user's exercise posture or physical condition;
a display panel displaying necessary information to a user during exercise based on the state information measured by the user state information measurement unit;
an exercise unit allowing a user to perform a resistance exercise or a balance exercise;
a coaching engine receiving and analyzing the state information measured by the user state information measurement unit during a user's exercise using the exercise unit and providing AI-based interactive coaching information to a user through the display panel; and
an expert terminal providing rehabilitation service information to a user exercising using the exercise unit through the display panel in parallel with the coaching engine.

2. The interactive AI coaching-based musculoskeletal exercise and rehabilitation training system according to claim 1, wherein the user state information measurement unit comprises:
a motion detector detecting a user's posture and motion;
a plantar pressure measurement unit measuring a user's plantar pressure;
a heart rate measurement unit measuring a user's heart rate; and
a load detector detecting a load applied to the exercise unit by a user.

3. The interactive AI coaching-based musculoskeletal exercise and rehabilitation training system according to claim 1, wherein the exercise unit comprises at least one of:
a first resistance exercise device allowing a user to perform a first resistance exercise; and
a first balance exercise device allowing a user to perform a first balance exercise.

4. The interactive AI coaching-based musculoskeletal exercise and rehabilitation training system according to claim 3, wherein the first resistance exercise device comprises a wire pulley unit generating resistance against pulling force applied to a wire by a user.

5. The interactive AI coaching-based musculoskeletal exercise and rehabilitation training system according to claim 4, wherein the wire pulley unit comprises:
a rotor rotatably mounted in a housing securely supporting the display panel, the rotor being connected to one end of the wire; and
a drive unit generating resistance against pulling force applied to the wire by a user and rotating the rotor to allow the one end of the wire to be wound around the rotor when the pulling force is removed from the wire.

6. The interactive AI coaching-based musculoskeletal exercise and rehabilitation training system according to claim 4, wherein the first resistance exercise device further comprises a pair of arm units allowing adjustment of a position and angle of the wire pulled by a user in front of the display panel.

7. The interactive AI coaching-based musculoskeletal exercise and rehabilitation training system according to claim 6, wherein each of the pair of arm units comprises:
a rotary block disposed on one side of a housing securely supporting the display panel, the rotary block being coupled to the housing to be rotatable about a hollow vertical shaft through which the wire passes;
an arm mount coupled to the rotary block to be rotatable about a hollow horizontal shaft through which the wire passes; and
an arm member having one end coupled to the arm mount and provided in the form of a bar having a hollow inside to allow passage of the wire therethrough.

8. The interactive AI coaching-based musculoskeletal exercise and rehabilitation training system according to claim 4, wherein the exercise unit further comprises: a second resistance exercise device allowing a user to perform a second resistance exercise, the second resistance exercise device comprising:
a support placed on a floor in front of the display panel;
a footrest coupled to the support and securing a user's feet thereto;
a seat mounted on the support to be slidable back and forth and allowing a user to be seated thereon; and
a handlebar coupled to the wire and pulled by a user.

9. The interactive AI coaching-based musculoskeletal exercise and rehabilitation training system according to claim 3, wherein the first balance exercise device comprises a plantar pressure pad allowing a user to stand thereon and comprising a plantar pressure measurement unit measuring a user's plantar pressure, the plantar pressure pad further comprising a guidance indicator synchronized with information displayed on the display panel and indicating a position at which a user's feet are to be placed on the plantar pressure pad.

10. The interactive AI coaching-based musculoskeletal exercise and rehabilitation training system according to claim 3, wherein the exercise unit further comprises: a second balance exercise device allowing a user to perform a second balance exercise, the second balance exercise device comprising a guide beam unit delivering a guide beam to guide the user to stay within a detection range of a motion detector while the motion detector detects the user's posture and motion during the second balance exercise.

11. The interactive AI coaching-based musculoskeletal exercise and rehabilitation training system according to claim 1, wherein the coaching engine comprises:
a UI/UX providing unit allowing selection between a resistance exercise mode and a balance exercise mode using the display panel;
a network input/output unit allowing access to the cloud server; and
a curation unit evaluating a user's exercise based on the user state information received from the user state information measurement unit and curating a training program.

12. The interactive AI coaching-based musculoskeletal exercise and rehabilitation training system according to claim 11, wherein the coaching engine further comprises:
a training information analysis unit analyzing compatibility of the training program curated by the curation unit with the user state information received from the user state information measurement unit;
a training program preparation unit preparing an AI-based training prescription based on analysis by the training information analysis unit; and
a training coaching unit performing training coaching according to the training prescription prepared by the training program preparation unit.

13. A training method using the interactive AI coaching-based musculoskeletal exercise and rehabilitation training system according to claim 1, the training method comprising:
inputting user information;
selecting between a resistance exercise mode and a balance exercise mode;
performing a rehabilitation exercise;
measuring the state information during the rehabilitation exercise; and
receiving the AI-based interactive coaching information during the rehabilitation exercise.

14. The training method according to claim 13, further comprising: before receiving the AI-based interactive coaching information,
setting whether to receive the AI-based interactive coaching information during the rehabilitation exercise.

15. The training method according to claim 13, further comprising:
further receiving the rehabilitation service information from an expert during the rehabilitation exercise.
